(19) United States
(12) Patent Application Publication
(10) Pub. No.: US 2009/0060946 A1
(43) Pub. Date: Mar. 5, 2009

(54) Activation of antigen-specific T cells by virus/antigen-treated dendritic cells

(76) Inventor: Thorsten Ahlert, Heidelberg (DE)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/288,553

(22) Filed: Oct. 21, 2008

Related U.S. Application Data
(63) Continuation of application No. 10/110,442, filed on May 21, 2002.

(30) Foreign Application Priority Data
Oct. 13, 1999 (EP) 99 119 980.3

(51) Int. Cl. A61K 39/12; A61P 31/00

(52) U.S. Cl. 424/204.1

(57) ABSTRACT

The present invention relates to a T cell activating agent containing dendritic cells (DC) treated with virus-treated antigen and/or dendritic cells treated separately with virus and antigen, which may be used as a vaccine to stimulate an immune response in a patient obtainable by the activation of antigen-specific T cells (TC) in vivo, to a composition containing activated TC which are activated by the T cell activating agent in vitro, to a pharmaceutical composition containing the T cell activating agent and/or the composition as well as to methods for their production.

### CROSS REFERENCE TO RELATED APPLICATIONS

The present application is a continuation application of U.S. application Ser. No. 10/110,442, filed May 21, 2002, entitled ACTIVATION OF ANTIGEN-SPECIFIC T CELLS BY VIRUS/ANTIGEN-TREATED DENDRITIC CELLS, which is hereby incorporated by reference herein, which was a §371 national phase filing of PCT/EP00/10019 filed Oct. 11, 2000, and claims priority to European application No. EP 00 119 980.3 filed Oct. 13, 1999.

### DETAILED DESCRIPTION OF THE INVENTION

The present invention relates to a T cell activating agent containing dendritic cells (DC) treated with virus-treated antigen and/or dendritic cells treated separately with virus and antigen, which may be used as a vaccine to stimulate an immune response in a patient obtainable by the activation of antigen-specific T cells (TC) in vivo, to a composition containing activated TC which are activated by the T cell activating agent in vitro, to a pharmaceutical composition containing the T cell activating agent and/or the composition as well as to methods for their production.

The immune system of cancer patients but also of patients suffering from chronic infectious diseases, autoimmune diseases, renal failure with the need of dialysis, or inherited immune dysfunctions works inefficient and needs external help. The causes for this immune deficiency may be the diseases themselves or externally induced defects including immunosuppression by conventional cancer therapies. Furthermore, the recognition of diseases like cancer or infections by the immune system may face obstacles such as weak or inefficient presentation of disease-specific antigen.

Thus, reconstitution of immunocompetence for specific antigens is an object of intense research. Cell therapy by transfer of in vitro-activated allogeneic or autologous antigen-specific T cells or in vivo induction of such cells by vaccination procedures are current approaches aiming to solve the above-mentioned problems of immune deficiency.

T cells are lymphocytes which are able to provide specific and nonspecific immunologic help for several immune mechanisms and which may also directly attack and eradicate foreign or disease-associated cellular antigens. They are able to develop and/or transfer immunologic memory over months and years towards such antigens and thus, are important mediators of long-term immunologic protection.

The first approaches developed to confer an improved competence to a patient suffering from immune deficiency were adoptive cell transfer therapies. These therapies were mainly used for the treatment of cancer and employed in the first instance the so-called lymphokine-activated killer cells (LAK) and later lymphokine-activated tumor-infiltrating lymphocytes (TIL) for autologous transfer. The term “autologous” means that the cells which were transferred originated from the patient him- or herself. The killer cells were generated from peripheral blood or from tumor tissue which was freshly obtained by operation. The obtained killer cells were cultivated and activated mainly in medium containing interleukin 2 (IL-2), a T cell growth factor [refs. 1., 2.]. Other attempts of adoptive autologous and allogeneic cell transfer include stimulation of T cells with tumor cells and/or antibodies or cytokines in vitro or in vivo before they are adoptively transferred to the recipient [refs. 3. to 7.]. The term “allogeneic” means that the transferred cells originated from a nonidentical individual.

The use of dendritic cells (DC) for the in vivo or in vitro activation of antigen (peptide)-specific T cells has been established in very recent years [refs. 8. to 13.]. DCs are “professional” antigen-presenting cells which can be more powerful antigen-specific activators of T cells than the antigen-bearing cells themselves. DC can be pulsed or loaded with antigens such as peptides or cell lysates, for example antigens derived from tumor cells. The DCs process the antigenic material and integrate the products into MHC class I and/or class II complexes which are able to present them to T cells. For a full activation of killer T cells, a presentation of processed material in both class I and II MHC complexes is necessary, although the killer cells themselves are only able to recognise a presentation via MHC class I. MHC class II is necessary for the additional activation of helper T cells. This finding has led to the addition of substitutes like Keyhole-limpet haemocyanin (KLH) which represent MHC class II integratable helper antigens. Furthermore, such a substitute would be able to represent a neoantigen and thus, can serve as a tracer molecule [ref. 8.].

Newcastle Disease Virus (NDV) is an avian paramyxovirus which has been used for a long time in cancer therapy. This virus can be used directly for infection and lysis of cancer cells in vitro or in vivo, and it can be used for modification of tumor cells in vaccines in order to give rise to an improved adhesion of stimulatory cells to their target cells. NDV may also induce a broad range of co-stimulatory signals for T cell activation when it is used for modification in cellular vaccines [refs. 14. to 22.].

However, the above-mentioned approaches display several problems and disadvantages.

In case of the use of cytokines for the activation and expansion of immune cells in vitro, the methods which have been used previously have not shown acceptable clinical risk-benefit relations. This is mostly due to an unspecific activation of the whole immune system and a dependency of the transferred cells on in vivo cytokine substitution after the application to the patient. The resulting treatment of patients with high-dose cytokines is accompanied by significant side effects which have led to an abandonment of this approach. More sophisticated methods using an antigen-specific component or a T cell receptor trigger for in vitro activation of T cells in addition to low-dose cytokines yielded cells which showed only a limited activity. Usually, the activity of the cells is readily suppressed after transfer into the patient, or the immune cells do not find (i.e. do not migrate to) the targeted cells in vivo. Therefore, the immunologic memory generated in this way is only short-lived and, moreover, leads to early disease progression after occasional therapeutic success.

Until now, DCs have been used clinically for in vivo induction of antigen-specific immune responses. However, obstacles which prevented a successful therapy using DCs have been the generation and/or isolation of a sufficient amount or functionally active DCs for therapeutic application. The strategies which have been developed until now, have rarely considered the possibility of a tolerance induction by insufficiently pulsed or insufficiently differentiated DCs. Furthermore, the in vivo generation of T cell responses with DCs can be difficult in patients with a deficient immune system.

The in vivo oncolysis using NDV has turned out to be difficult because of an efficient inactivation of the virus by the patient's immune system and because of virus-resistant tumor cells in heterogeneous tumors. Virus-modified tumor cell vaccines which have been used until now for in vivo activation of antigen-specific T cells show only limited effects which are only observed in early cancer stages. Approaches using NDV face further obstacles such as immune suppressed or immune deficient patients, insufficiently active T cells because of an antigen overload in the vaccinated patient or because of inhibitory factors which are produced by the target cells for T cells. Furthermore, suboptimal antigen presentation on antigen-varying target (i.e., in this case, tumor) cells and on in vivo preexisting antigen-presenting cells can be the reason for the failure of T cell activation.

A further problem of virus-modified cell vaccines which have been developed until now is that they need a significant number of viable antigen-bearing cells in order to reach a sufficient efficiency. This has limited the use of virus-modified cell vaccines in clinical applications so far because in clinical situations a comparably large amount of raw material (mostly surgically resectable tumor material) is available which contains considerable amounts of dead cells.

Moreover, the necessity of malignant viable cells in virus-modified vaccines results in the need of irradiation of the cells for their inactivation before in vivo use. This inactivation method is effective, however, it is difficult to apply with respect to the controlled pharmaceutical production process [refs. 20., 23. to 25.].

Therefore, the technical problem underlying the present invention is to improve the in vivo and in vitro induction of highly active antigen-specific immune stimulators, the effect of which is especially mediated by T cells and T memory cells during therapeutic clinical use. This improvement includes the reduction of the possibility of tolerance induction by T cell activation efforts and, thus, increasing the safety of the procedure.

The solution of the above technical problem is achieved by providing the embodiments as characterized in the claims.

In particular, the present invention relates to a composition containing activated T cells which are capable of performing and stimulating a specific immunoresponse in a patient, the T cells being prepared from the patient or a relative thereof and activated by treatment with a T cell activating agent in vitro, wherein the T cell activating agent comprises activated dendritic cells which are activated by the method comprising the steps of
(a) preparing an antigen and optionally treating the antigen with a virus in vitro,
(b) preparing monocytes from the patient or a relative thereof,
(c) developing dendritic cells from the monocytes by incubation in vitro,
(d) coincubating the obtained dendritic cells with the virus-treated antigen obtained in step (a) in vitro and/or with the untreated antigen obtained in step (a) plus the virus in vitro,
   wherein the virus is capable of improving the adhesion of the antigen to and the presentation of the antigen by the dendritic cells and which is capable of modulating the activation, maturation, stability and cosignalling of the dendritic cells.

The term “T cell activating agent” as used herein means a composition or formulation containing activated dendritic cells which are capable of stimulating an immune response in a patient against antigens. The activated dendritic cells of the above-defined T cell activating agent are capable of activating T cells in vivo as well as in vitro.

Therefore, according to one aspect, the present invention relates to the above-defined composition in which the T cell activating agent as characterized above is used for T cell activation in vitro. According to a further aspect, the present invention relates to the T cell activating agent itself which may be used as a vaccine for T cell activation in vivo.

Thus, the present invention provides novel systems for the improvement of both in vivo and in vitro induction of highly active, antigen-specific immune stimulators which may be used in the two following different therapeutic regimens:
(1) The composition of the present invention is particularly useful in a method of cellular therapy, wherein the activated T cells are administered to the patient. Such a method is also referred to as adoptive or passive immunotherapy (ADI).
(2) The T cell activating agent according to the present invention provides a vaccine, e.g. a tumor vaccine, for immunizing a patient with an antigen such as a tumor antigen in a highly immunogenic form. Such a therapeutic regimen is also referred to as active specific immunotherapy (ASI).

For stimulating T cells in vivo, the activated dendritic cells may be administered, e.g. intracutaneously, subcutaneously or intralymphatically, to the patient, and patient's T cells migrate to the administration locus where they are activated by the dendritic cells. The T cell activating agent used for the activation of T cells in the composition according to the present invention may also contain substances which are prepared using recombinant DNA technology. Preferably, the T cell activating agent used for T cell activation in the composition according to the present invention contains one or more other T cell activating agents which act additively or synergistically with the dendritic cells.

The term “antigen” comprises any structure which is capable of inducing an immune response in an organism either by itself or when coupled to a suitable carrier molecule or cell. Therefore, antigens according to the present invention include low molecular compounds which serve as haptens as well as whole cells such as tumor cells as well as the parts thereof such as polypeptides, oligopeptides derived therefrom, lipids such as glycolipids, polysaccharides and nucleic acids. Further antigens according to the present invention are viruses as well as their parts and any prokaryotic organism such as bacteria as well as eukaryotic organisms. According to a preferred embodiment of the above-defined composition, the antigen is prepared from the patient, however, as defined above, the antigen may as well be prepared from other organisms or may be synthetic or biosynthetic.

Preferably, the antigen which may be virus-treated in step (a) such as virus-treated living tumor cells may be inactivated without the use of irradiation prior to the coincubation with the dendritic cells in step (d) of the above-defined method. Preferred methods for inactivation and lysis of living cells such as living tumor cells include, for example, freeze-thawing and ultrasonification. The use of methods apart from irradiation poses less problems to the pharmaceutical production process.

The use of bone marrow in addition or instead of blood as the source for T cells which are to be activated leads to an increase of the yield of highly active antigen-specific T cells, for example T memory cells, for the stimulation by coincubation with NDV-modified DCs.

Preferably, the antigen such as a cell, e.g. a tumor cell, may be further purified during the preparation from the patient, for example by immunobead techniques. These techniques comprise the use of small magnetic metal beads (e.g. from Dynal or Milteny) which are coupled to antibodies directed against contaminating components such as cells or other agents. After having bound to the antibody-coupled beads during an incubation step, the contaminations are removed from the T cell activating agent, e.g. a suspension of the activated dendritic cells, by applying a magnetic field which draws the beads out of the suspension. Further, the cells may be cryoconservated after their preparation, e.g. from the patient, in step (a) above and may be thawed before or after treatment with the virus in step (a) and/or coincubation with the dendritic cells in step (d) above.

Since dendritic cells are capable of processing antigens derived from genetic material, it is also possible to use genetic material, i.e. a nucleic acid such as DNA or RNA (preferably mRNA), encoding the virus-treated/modified antigen and/or the immunological signals said virus-treated/modified antigen provides, for activating (pulsing) dendritic cells instead of or in addition to the antigen itself in step (d) of the method for DC activation as defined above. For example, the dendritic cells may be treated with the corresponding nucleic acid(s) by transfection (e.g. using Ca-phosphate, lipofection or electroporation methods). Preferred sources of the nucleic acid(s) are virus-infected antigen presenting cells. These cells process not only gene products for antigen expression, but also products, e.g. a cocktail of cytokines, heat shock proteins etc., induced by virus infection serving as immunological signals. For example, the mRNA coding for such products may be transcribed into DNA and thereafter this genetic material may be amplified by the use of PCR. Thus, a constant source of virus-treated/modified antigen for continued treatment of large numbers of patients can be provided which is pharmaceutically easy to handle.

Preferably, the developing dendritic cells are also coincubated with the virus during the step of incubation in vitro (c).

According to further preferred embodiments of the above-defined composition and the T cell activating agent, the virus used is selected from the group consisting of paramyxoviruses such as Newcastle disease virus (NDV) or mumps virus, vaccinia virus, myxovirus, herpesvirus, AIDS virus, human papillomavirus (HPV) and mouse mammary tumor virus (MMTV).

The T cell activating agent used for T cell activation in the composition according to the present invention comprises dendritic cells which are activated with a virus and an antigen which is preferably prepared from a patient having a significantly impaired immune system. Preferably, this impairment of a patient's immune system may be caused by chronic disorders such as cancer, infections, renal failure which has to be treated by dialysis, autoimmune diseases and/or inherited immune dysfunctions.

The term “patient” as used herein comprises humans as well as animals. The preferred patient is a human. The “relative” of the patient is a person or animal, respectively, being related by blood and/or genetically via HLA-type with the patient, i.e. the human or animal.

In the above-defined composition the T cells are activated by the method comprising the steps of
(i) preparing T cells from the patient or relative thereof, and
(ii) treating the T cells with the T cell activating agent as defined above in vitro.

Preferably, the treatment of T cells with the T cell activating agent according to the present invention in step (ii) above is carried out by coincubation in a low- or medium-dose cytokine-containing medium for a short time. More preferably, the culture medium contains not more than 6000 U/ml of cytokines, for example IL-2, and the cultivation in the low-dose cytokine-containing medium is not longer than seven days.

According to preferred embodiments of the T cell activating agent and the composition according to the present invention at least part of the monocytes prepared in step (b) of the above-defined T cell activating agent and at least part of the T cells prepared in step (i) of the above-defined composition may be derived from the patient's or relative's bone marrow or blood. Therefore, in contrast to prior art cell therapy vaccines, bone marrow may be used in the above-defined T cell activating agent as a very efficient source of monocytes from which dendritic cells are developed and, furthermore, in the above-defined composition as a very efficient source of (memory) T cells which are obtained for in vitro activation with virus-treated DCs in addition to monocytes or T cells, respectively, from peripheral blood.

In addition to the above-mentioned advantages of the T cell activating agent and the composition according to the present invention, they show several further advantages due to the novel approach for the activation of T cells.

1,5×10⁶ NDV-modified DCs and an equivalent of 1,5×10⁶ target cells (for example tumor cells) when used as the antigen are needed for human in vivo vaccination or for a reasonably effective in vitro stimulation of T cells derived, for example, from humans. Taking these considerations into account the method for T cell activation as described above provides the possibility to use target cells as antigens which may be either dead or alive, since the uptake and processing of the material by the DCs leads to an antigen presentation to living TCs in the end. In contrast, in conventional virus-modified tumor cell vaccines at least 1,5×10⁶ target cells must be alive in order to lead to an efficient T cell activation and no more than 66% of dead cells should contaminate the living target cells [refs. 20., 24. to 26.]. However, an average of 50% of the target cells are dead in, for example, fresh tumor cells suspensions after cryoconservation which is mostly necessary for the storage of the raw material. Therefore, the use of NDV-modified DCs instead of original antigen-bearing living target cells reduces about 50% of the amount of raw material needed, since dead target cells as well as living target cells can be used as the antigen (the living target cells are preferably disintegrated by shock freezing or by the infection with the virus).

Furthermore, the use of a virus, for example NDV, in order to increase the DC number and to enhance their function facilitates and increases the generation of efficient DCs which can be used for in vitro or in vivo activation of (memory) T cells.

The use of a virus, for example a paramyxovirus such as NDV, which is capable of improving the adhesion of the antigen to the dendritic cells and which is capable of stimulating the activation of the dendritic cells as described above further reduces the probability of an induction of tolerance by an inefficient number and/or function of DCs in the patient. This advantage and the fact that the use of a virus as described above for increasing the number of DCs as well as their function improves and increases the generation of efficient DCs represent further surprising properties of the T cell activating agent according to the present invention which can not be predicted from known properties of viruses such as NDV in tumor cell modification. Generally, DCs per se should be able to perform an optimal antigen presentation function and a costimulatory signalling for T cell activation. Therefore, in theory, there is no obvious need for the effects of a virus like NDV on DCs. However, while a virus such as NDV in fact induces a secretion of costimulatory cytokines and provides adhesion molecules for longer T cell-target cell interaction for the preparation of cellular vaccines such as the above described tumor cell vaccines, it improves the maturation and/or differentiation and/or antigen presentation, respectively, of DCs. Furthermore, the virus such as NDV may also modify in addition or instead of inducing a costimulatory signalling in DCs in order to generate an improved T cell activation.

Also, according to preferred embodiments of the composition and the T cell activating agent according to the present invention, the virus such as NDV is capable of inducing at least in part fusions between the antigen and the dendritic cells in step (d) of the activation of the dendritic cells. The fusion may be mediated via the virus' fusion protein leading to hybrids between the dendritic cells and virus-treated antigen such as a cell. Such hybrids further improve the antigen-presenting capability and T cell activating activities of dendritic cells. The antigen is preferably a cell such as a tumor cell derived from a patient or a cell derived from a tumor cell line which confers the hybrid with multiple tumor-associated antigens.

These effects on DCs were not expected from the known properties of viruses such as NDV on tumor cells and tumor cell vaccines. On the contrary, one would have expected from prior art studies that viruses suppress DCs. For example, Jenne et al. [ref. 27.] found a suppression of T cell stimulation properties by viruses and Raftery et al. [ref. 28.] found changes in DC function which were believed to contribute to human cytomegalovirus-associated immunosuppression after infection of DC with human cytomegalovirus.

Furthermore, the T cells which are contained in the composition according to the present invention and which are activated by the above-described method using the above-defined T cell activating agent in vitro, exhibit a high efficiency and reduced dependency on in vivo application of cytokines which reduces potential side-effects of a therapy using the composition according to the present invention. Moreover, the T cells activated by the method as described above show a reduced sensitivity to inactivating mechanisms in a patient, since the T cells activated according to the present invention are more differentiated and more efficiently activated.

A further embodiment of the present invention relates to a pharmaceutical composition containing a pharmaceutically effective amount of the T cell activating agent and/or the composition according to the present invention, optionally in combination with a pharmaceutically acceptable carrier and/or diluent, preferably for the curative or prophylactic treatment of cancer, infections and autoimmune diseases. Furthermore, the pharmaceutical composition according to the present invention may contain one or more other T cell activating agents which may act additively or synergistically with the T cell activating agent and/or composition according to the present invention. The pharmaceutical composition according to the present invention may be applied by any conventional application route used in vaccination or cell therapy such as intravenous, intramuscular, intracutaneous, subcutaneous and/or intralymphatic administration, for example by infusion or injection.

The pharmaceutical composition according to the present invention may be applied in a method for the treatment of a patient suffering from an impairment of the immune system which may be caused by disorders such as by cancer, infections, renal failure, autoimmune diseases and/or inherited immunodysfunctions comprising the step of administering the above-defined pharmaceutical composition in an amount sufficient to stimulate and/or to perform a specific immunoresponse in the patient.

Further embodiments of the present invention relate to methods for the preparation of activated dendritic cells and activated T cells, respectively. The method for the preparation of activated dendritic cells according to the present invention comprises the steps of
(a) preparing an antigen and optionally treating the antigen with a virus in vitro,
(b) preparing monocytes from the patient or a relative thereof,
(c) developing dendritic cells from the monocytes by incubation in vitro,
(d) coincubating the obtained dendritic cells with the virus-treated antigen obtained in step (a) in vitro and/or with the untreated antigen obtained in step (a) plus the virus in vitro,
   wherein the virus is capable of improving the adhesion of the antigen to and the presentation of the antigen by the dendritic cells and which is capable of modulating the activation, maturation, stability and cosignalling of the dendritic cells.

The method for the preparation of activated T cells according to the present invention comprises the steps of
(i) preparing T cells from a patient or relative thereof,
(ii) treating the T cells with the above-defined T cell activating agent in vitro.

The present invention will be further illustrated by the following non-limiting example.

### EXAMPLE

### Adoptive Cell Therapy with Allogeneic TCs which have been Activated In Vitro with NDV-Modified DCs Loaded with Tumor Cell Material

Preparation of the Antigen from the Patient

The preparation of NDV-modified tumor cells comprises the following steps:
(1) Isolation of tumor material by surgical intervention
(2) Dissociation of tumor cell material into a suspension of single cells by mechanical and enzymatic means: four times incubation for 30 min at 37° C. with collagenase (5 U/ml) and DNase (15 U/ml). Optionally, immunobead purification of tumor cells which reduces contaminating non-tumor cells.
(3) Cryoconservation of tumor cells
(4) Thawing of tumor cells and modification/infection with NDV: 20 to 100 hemagglutinating units of virus per 1×10⁷ cells.
(5) Inactivation of NDV-modified tumor cells by 4 to 5 cycles of shock freezing and thawing.
   Preparation of T Cells and Dendritic Cells from a Relative of the Patient
(1) Taking bone marrow and/or blood from the relative
(2) Preparing monocytes from the bone marrow and/or blood and inducing maturation and differentiation into DCs by standard cultivation with interleukin-4 (IL-4), granulocyte macrophage colony stimulating factor (GM-CSF) and tumor necrosis factor (TNF) with and without NDV
   (i) isolation of cells from 150 ml blood
   (ii) cultivation of monocytes for one day in RPMI 1640 plus 2 mM glutamine, 100 U/ml penicillin, 100 μg/ml streptomycin and supplemented with 5% autologous, noninactivated plasma, 1000 U/ml IL-4 (from Promocell) and 1000 U/ml GM-CSF
   (iii) change of medium and cultivation for two days in the above medium (day 1)
   (iv) change of medium, addition of TNFα (from Promocell) at a final concentration of 10 ng/ml (day 4)
(3) Loading/Pulsing of DCs with virus-modified tumor cell lysate by adding the lysate to the DCs followed by incubation in cell culture medium
   (v) addition of DCs to tumor cells at a ratio of 1 part DCs to 3 parts dead tumor cells in 1 ml X-vivo medium, centrifugation at low speed [1000 revolutions per hour (rph)], incubation at 37° C. for 4 h (day 5)
   (vi) control of antigen-loaded DCs using fluorescence-activated cell sorting (FACS) analysis
   (vii) cultivation of antigen-loaded DCs in RPMI+5% plasma+IL-4+GM-CSF+TNFα for three days (until day 8)
(4) Isolation of TCs from bone marrow and/or blood by a method comprising an immunobead enrichment step (this may be carried out during the generation/loading phase of the TCs)
   (viii) isolation of TCs from 150 ml blood (erythrocyte lysis, adherence, Pan T cell isolation kit)
   (ix) control of TCs using FACS analysis
(5) Expansion of TCs in cell culture
(6) Coincubation of TCs and DCs which have been pulsed with NDV-modified tumor lysate which comprises a short incubation in the presence of cytokines at low- or medium-dose in order to avoid induction of dependency of the TCs on these cytokines
   (x) addition of TCs to the antigen-loaded DCs in a ratio of 1 part DCs to 34 parts TCs and incubation in fresh RPMI supplemented with 5% plasma (but without cytokines) for three days (until day 11)
   (xi) change of medium and addition of IL-2 (6000 U/ml) on day 11 and cultivation for three days
   (xii) harvesting the activated TCs, resuspending the TCs in 10 ml medium I, filling the suspension in a syringe for intravenous injection, filtration; the filtrate is taken up in 400 μl medium I and injected subcutaneously.
(7) Analysis of antitumor activity of the activated TCs using ELISPOT
   T cells are coincubated (challenged) with antigen for 20 h.

Thereafter, γ-interferon (IFN-γ) production is detected for each single T cell on a plate coated with anti-IFN-γ antibodies. Bound IFN-γ is detected in spots surrounding the T cells by means of ELISA stain.

### Therapy

### (1) Day 1 to 14

Immunosuppression of the patient with medium-dose or dose-intensified chemotherapy and/or radiotherapy and/or corticosteroids and cyclosporin, which is necessary in order to avoid the rejection of the allogeneic DC-NDV tumor-actived TCs of the patient's relative.
The patient is treated with 80 mg/m² taxol, 40 mg/m² epirubicin and 50 mg of hydrocortisol per day for two consecutive weeks. In addition, 30 Gray irradiations of a bone metastasis were carried out.

### (2) Day 15

Intravenous infusion of the allogeneic TCs which were activated by NDV-DC treatment
About 5×10⁸ T cells which have been activated by the above-described method are infused in a volume of 250 ml Ringer lactate solution.
   (3) Next cycle of treatment after a break of six weeks.

### Materials

Recombinant human (rHU) IL-4 cc dissolved in phosphate buffered saline (PBS)/1% human serum albumin (HSA) (stock solution: 1×10⁵ U/ml, corresponding to 1×10⁵ μg/ml)

GM-CSF dissolved in PBS/1% bovine serum albumin (BSA) (stock solution: 1×10⁵ U/ml)

rHU TNFα dissolved in RPMI 1640/1% BSA (stock solution: 1 μg/ml)

IL-2 dissolved in X-vivo (stock solution: 6×10⁵ U/ml, diluted 1:100), proleukin (from Chiron)

Increased T Cell Activating Properties of Dendritic Cells when Pulsed with Virus-Treated Antigen Versus Non-Treated Antigen

### Virus-Treated Versus Non-Treated Antigen

MCF-7 cells were cultured in RPMI medium, supplemented with 10% fetal calf serum (FCS). 1×10⁷ cells were washed in order to remove FCS and infected with 60 Hemaglutinating Units of Newcastle Disease Virus strain Ulster in RPMI medium by adding virus solution for 60 min at 37° C. Non-adsorbed virus were washed-off again before an incubation for 24 h at 37° C. in RPMI/2% FCS was carried out.

Control cells were not infected with virus but otherwise treated in the same way as infected cells (i.e. incubation for 60 min in RPMI without FCS followed by incubation for 24 h in RPMI/2% FCS).

After the incubation was completed, infected (MCF-7-NDV) and control cells were lysed by three cycles of freeze-thawing. Protein content was estimated in both preparations.

### Preparation of Dendritic Cells

(1) Taking bone marrow from a breast cancer patient
(2) Preparing dendritic cells from the bone marrow as described above under item (2) of “preparation of T cells and dendritic cells from a relative of the patient”.
   Loading/Pulsing Dendritic Cells with Antigen

1×10⁶ dendritic cells were coincubated with 200 μg/ml lysed MCF-7-NDV or with 200 μg/ml lysed non-infected MCF-7-cells. This was carried out by washing dendritic cells and adding the washed cells to the corresponding antigenic protein solution.

Activation of T Cells with Antigen-Pulsed Dendritic Cells

Autologous T cells from the patient were prepared from bone marrow as described above under item (4) of “preparation of T cells and dendritic cells from a relative of the patient”. Antigen-pulsed dendritic cells were added to the T cells in a ratio of one dendritic cell to five T cells. Incubation was carried out for 48 h.

Determination of Anti-Tumor Memory T Cell Response with ELISPOT

Activated T cells were determined on a single cell basis by their γ-interferon production using the ELISPOT assay. Bound γ-interferon is detected in spots surrounding the T cells by means of an ELISA stain as described above under item (7) of “preparation of T cells and dendritic cells from a relative of the patient”.

### Results

415 spot forming cells in 2,5×10⁴ T cells were detected after simulation with MCF-7-NDV-pulsed dendritic cells. Only 190 spot forming cells were dected in 2,5×10⁴ T cells stimulated with non-infected MCF-7 cells. 150 spot forming cells were detected in 2,5×10⁴ T cells stimulated with non-pulsed dendritic cells. Less than 12 spots were observed in 2,5×10⁴ T cells not stimulated at all. 165 spots were counted when dendritic cells had been pulsed with a non-breast cancer cell line.

### CONCLUSION

The above results show that in the breast cancer patient the T cell stimulatory capacity of the dendritic cells which had been pulsed with virus-infected antigen was more than doubled in comparison to dendritic cells which had been pulsed with non-infected antigen, non-pulsed dendritic cells and dendritic cells which had been pulsed with a non-breast cancer cell line.

Stability of Increased Stimulating Properties of Dendritic Cells which have been Pulsed with Virus-Infected Tumor Cells

Irradiated MCF-7 tumor cells used as antigen were infected for 30 min with NDV and stored overnight at 4° C. without further incubation.

As a control, non-infected MCF-7 cells were used which were otherwise treated in the same way as NDV-infected cells. As a further control, peripheral blood leukocytes were used.

Dendritic cells were generated by incubation of monocytes from peripheral blood of a breast cancer patient with GM-CSF and interleukin-4 (IL-4) for 5 days using a standard protocol (cf., for example, ref. 8.).

Dendritic cells were pulsed with infected, irradiated but non-lysed MCF-7 cells or control cells by coincubation at 37° C. for 6 h in medium without cytokines. After 6 h TNF-α, IL-1, IL-6 and prostaglandin E2 were added to the cultures in order to support final differentiation of dendritic cells. Thereafter, incubation was continued for 40 h.

Pulsed dendritic cells were washed in order to remove cytokines. The washed cells were stored at 4° C. for 6 h, followed by incubation for 90 h at 37° C. in medium containing autologous serum but no cytokines. This procedure imitates storage of a vaccine at 4° C. and then in vivo persistence of pulsed dendritic cells in the autologous patient after injection of the vaccine.

After 90 h the antigen-pulsed dendritic cells were used for short term stimulation (42 h) of autologous T cells purified from peripheral blood of the patient. The ratio of dendritic cells to T cells was from 1 to 10 to 1 to 100.

After 42 h of short term stimulation γ-interferon production (activation) in T cells was determined by the above-described ELISPOT method.

### Results

Dendritic cells, pulsed with virus-infected antigen (MCF-7 tumor cells), induced substantially more γ-interferon producing (i.e. activated) T cells than those dendritic cells which had been pulsed with control cells. Furthermore, the dendritic cells pulsed with virus-infected MCF-7 cells stimulated T cells more efficiently than virus-infected MCF-7 cells alone, non-infected MCF-7 cells alone, virus-pulsed dendritic cells or dendritic cells pulsed with peripheral blood leucocytes. Thus, the effect of virus enhancement of dendritic cell stimulatory activities was stable even after more than 90 hours of incubation without cytokines. Therefore, a T cell activating agent used as a vaccine containing these cells is capable of maintaining its in vivo T (memory) cell stimulating activity for at least this time period.

### REFERENCES

1. Rosenberg S A, Lotze M T, Muul L M, Chang A E, Avis F P, Leitmann, Linehan W M, Robertson C N, Lee R E, Rubin J T, Seipp C A, Simpson R N, White D E: A progress report on the treatment of 157 patients with advanced cancer using lymphokine-activated killer cells and interleukin-2 or high-dose interleukin-2 alone. N Engl J Med 316. (15) 889-897, 1987
2. Rosenberg S A, Packard B S, Aeberseld P M, Solomon D, Topalian SL, Toy S T, Simon P, Lotze M T, Vang J C, Seipp C A, Simpson C, Carter C, Bork S, Schwarzentruber D, Wei J P, White D E: Use of tumorinfiltrating lymphocytes and interleukin-2 in the immunotherapy of patients with metastatic melanoma. New Engl J Med 319. 1676-1680, 1988
3. Fichtner K-P, Schirrmacher V, Griesbach A, Hull WE: In Vivo 1H-NMR microimaging with respiratory triggering for monitoring adoptive immunotherapy of metastatic mouse lymphoma. Magnetic Resonance In Medicine (MRM) 38. 440-455, 1997
4. Yamagishi H, Ueda Y, Oka T: A case report of immunotherapy on a patient with advanced gastric cancer by adoptive transfer of OK-432 reactive HLA-matched allogeneic lymphocytes. Cancer Immunol Immunother 46. 113-119, 1998
5. Schirrmacher V, Beckhove P, Krüger A, Rocha M, Umanski V, Fichtner K-P, Hull W E, Zangemeister-Wittke U, Griesbach A, Jurianz K, Hoegen Pv: Effective immune rejection of advanced metastasized cancer. Int J Oncol 6. 505-521, 1995
6. Cardoso A A, Seamon M J, Afonso H M, Ghia P, Boussiotis V A, Freeman G J, Gribben J G, Sallan S E, Nadler L M: Ex vivo generation of human anti-pre-B leukemia-specific autologous cytolytic T cells. Blood 90. (2) 549-561, 1997
7. Tani M, Tanimura H, Yamaue H, Mizobata S, Iwahashi M, Tsunoda T, Noguchi K, Tamai M, Hotta T, Terasawa H, Arii K: Generation of CD4+ cytotoxic T-lymphocytes stimulated by immobilized anti-CD3 monoclonal antibody and interleukin-2 in cancer patients. Int J Cancer 60. 802-807, 1995
8. Nestle F O, Alijagic S, Gilliet M, Sun Y, Grabbe S, Dummer R, Burg G, Schadendorf D: Vaccination of melanoma patients with peptide- or tumor lysate-pulsed dendritic cells. Nature Medicine 4. (3) 328-332, 1998
9. Höltl L, Rieser C, Papesh C, Ramoner R, Herold M, Klocker H, Radmayr C, Stenzl A, Bartsch G, Thurnher M: Cellular and humoral immune responses in patients with metastatic renal cell carcinoma after vaccination with antigen pulsed dendritic cells. J Urol 161. 777-782, 1999
10. Hsu F J, Benike C, Fagoni F, Liles T M, Czerwinski D, Taidi B, Engleman E G, Levy R: Vaccination of patients with B-cell lymphoma using autologous antigen-pulsed dendritic cells. Nature Medicine 2. 52-58, 1996
11. Salgaller M L, Lodge P A, McLean J G, Tjoa B A, Loftus D J, Ragde H, Kenny G M, Rogers M, Boynton A L, Murphy G P: Report of immune monitoring of prostate cancer patients undergoing T cell therapy using dendritic cells pulsed with HSA-A2-specific peptides from prostate-specific membrane antigen (PSMA). Prostate 35. 144-148, 1998
12. Bennet SRM: Induction of a CD8− cytotoxic T lymphocyte response by crosspriming requires cognate CD4-help. J Exp Med 186. 65-70, 1997
13. Peters J H, Gieseler R, Thiele B, Steinbach F: Dendritic cells: from ontogenetic orphans to myelomonocytic descendants. Immunology today 17. (6) 273-278, 1996
14. Ahlert T and Schirrmacher V. Isolation of a human melanoma adapted Newcastle Disease Virus mutant with highly selective replication patterns. canc res 1990; 505962-8.
15. Ahlert T, Kaufmann M, Bastert G, Schirrmacher V.: Active Specific Immunotherapy (ASI) with virally modified autologous tumor cells in breast and ovarian cancer: factors influencing immunological vaccination success. J of Canc Res and Clin Oncol 116/supplement.: 80, 1990 (abstr)
16. Cassel W A, Murray DR: A ten-year follow-up on stage II malignang melanoma patients treated postsurgically with Neuwcastle disease virus oncolysate. Med Oncol & Tumor Pharmacother 9. 169-171, 1992
17. Cassel W A, Garret RE: Newcastle Disease Virus as an antineoplastic agent. Cancer 66. (7) 1517-1523, 1965
18. Csatary L K, Eckhard S, Bukosza I, Czegledi F, Fenyvesi C, Gergely P, Bodey B, Csatary CM: Attenuated veterinary virus vaccine for the treatment of cancer. Cancer Detection and Prevention 17. 619-627, 1993
19. Schirrmacher V, Haas C, Bonifer R, Ahlert T, Gerhards R, Ertel C: Human tumor cell modification by virus infection: an efficient and safe way to produce cancer vaccine with pleiotropic immune stimulatory properties when using Newcastle disease virus. Gene therapy 6. 63-73, 1999
20. Schirrmacher V, Ahlert T, Pröbstle T, Steiner H-H, Herold-Mende C, Gerhards R, Hagmüller E: Immunisation with virus modified tumor cells. Seminars in oncology 25. (6) 677-696, 1998
21. Schirrmacher V, Hoegen von P, Heicappell R: Virus modified tumor cell vaccines for active specific immunotherapy of mecrometastases: Expansion and activation of tumor-specific T cells. Immunity to Cancer 2. 391-399, 1989
22. Schirrmacher V, Ahlert T, Heicappell R, Appelhans B, Hoegen von P: Successful application of non-oncogenic viruses for antimetastatic cancer immunotherapy. Cancer Res 5. 19-49, 1986
23. Ahlert T, Sauerbrei W, Bastert G, Ruhland S, Bartik B, Simiantonaki N, Schumacher J, Häcker B, Schuhmacher M, Schirrmacher V: Tumor-cell number and viability as quality and efficacy parameters of autologous virus-modified cancer vaccines in patients with breast or ovarian cancer —Errata. Journal of clinical oncology 15. (4) 2763, 1997
24. Schirrmacher V, Griesbach A, Zangemeister-Wittke U: y-irradiated viable tumor cells as whole-celle vaccines can stimulate in situ syngenelc antitumor cytotoxic T lymphocytes and delayed-type hypersensitivity reactivity whereas tumor cell lysates elicit only delayed-type hypersensitivity reactivity. Vaccine Res 3—No. 1. 31-48, 1994
25. Schirrmacher V, Hoegen von P: Importance of tumor cell membrane integrity and viability for cytotoxic T lymphocyte activation by cancer vaccines. Vaccine Res 2—No. 3. 183-196, 1993
26. Ahlert T, Sauerbrei W, Bastert G, Ruhland S, Bartik B, Simiantonaki N, Schumacher J, Häcker B, Schumacher M, Schirrmacher V: Tumor cell number and viability as quality and efficacy parameters of autologous virus modified cancer vaccines. J Clin Oncol 15. 1354-1366, 1997
27. Jenne L et al., Immunobiology 200 (1999) 3-5, pp. 562
28. Raftery M et al., Immunobiology 200 (1999) 3-5, pp. 568-28

What is claimed is:

1. A method of preparing a T cell activating agent comprising activated dendritic cells, the method comprising the steps of:
(a) treating a cellular antigen with a virus in vitro; and
(b) co-incubating in vitro the virus-treated cellular antigen with in vitro differentiated dendritic cells to form a T cell activating agent.

2. The method according to claim 1, further comprising the step of inactivating the virus-treated cellular antigen obtained in step (a) without the use of irradiation.

3. The method according to claim 2, wherein the virus-treated cellular antigen is inactivated by freeze-thawing or ultrasonication.

4. The method according to claim 1, wherein the cellular antigen is prepared from a patient to whom the agent is later administered.

5. The method according to claim 1, wherein the cellular antigen is a tumor cell.

6. The method according to claim 1, wherein the cellular antigen has been purified by an immunobead technique.

7. The method according to claim 1, wherein the cellular antigen is cryopreserved after its preparation and thawed before or after the treatment with the virus in step (a).

8. The method according to claim 1, wherein the virus-treated cellular antigen from step (a) is cryopreserved after step (a) and thawed before or after co-incubation with the dendritic cells in step (b).

9. The method according to claim 1, wherein the dendritic cells are obtained by differentiation from monocytes and co-incubated with the virus during said differentiation.

10. The method according to claim 1, wherein the virus induces fusion at least in part between the cellular antigen and the dendritic cells in step (b).

11. The method according to claim 1, wherein the virus is selected from the group consisting of paramyxoviruses, vaccinia virus, myxovirus, herpesvirus, human immunodeficiency virus, human papillomavirus and mouse mammary tumor virus.

12. The method according to claim 11, wherein the virus is a paramyxovirus, and the paramyxovirus is Newcastle disease virus or mumps virus.

13. The method according to claim 4, wherein the patient's immune system is impaired.

14. The method according to claim 13, wherein the impairment of the immune system is caused by one or more of cancer, infection, renal failure, an autoimmune disease, or an inherited immune dysfunction.

15. The method according to claim 4, wherein the patient is a human.

16. The method according to claim 1, further comprising incubating said isolated dendritic cells in vitro with one or more nucleic acids coding for a protein component of said virus-treated cellular antigen.

17. A method of preparing a T cell activating agent comprising activated dendritic cells, the method comprising incubating isolated dendritic cells in vitro with one or more nucleic acids coding for a protein component of a cellular antigen to form a T cell activating agent.

18. A T cell activating agent produced according to the method of claim 1.

19. The T cell activating agent according to claim 18, further comprising another T cell activating agent.

20. A pharmaceutical composition comprising a pharmaceutically effective amount of the T-cell activating agent of claim 19 and a pharmaceutically acceptable carrier or diluent.
